(19) United States
(12) Patent Application Publication
(10) Pub. No.: US 2008/0213188 A1
(43) Pub. Date: Sep. 4, 2008

(54) NOVEL OPHTHALMIC COMPOSITIONS CONTAINING HUMAN RECOMBINANT LYSOZYME AND USE THEREOF FOR TREATING EYE CONDITIONS AND AS CONTACT LENS SOLUTIONS

(75) Inventor: STEFANO FERRARI, Rhinebeck, NY (US)

Correspondence Address: NORRIS, MCLAUGHLIN & MARCUS, PA, 875 THIRD AVENUE, 18TH FLOOR, NEW YORK, NY, 10022, US

(73) Assignee: SAINT SIMEON LDA, Rhinebeck, NY (US)

(21) Appl. No.: 12/039,922

(22) Filed: Feb. 29, 2008

Related U.S. Application Data
(60) Provisional application No. 60/892,565, filed on Mar. 2, 2007.

(51) Int. Cl. A61K 38/46; A61K 51/00; A61P 27/02

(52) U.S. Cl. 424/9.6; 424/94.61; 424/9.1

(57) ABSTRACT

An ophthalmic solution comprising: a) a human recombinant lysozyme; b) one or more natural lacrophyl substances; c) water; and d) optionally one or more therapeutic substances. The ophthalmic solution is useful to treat dry eye conditions and eye inflammation and also to condition and/or cleanse contact lenses. It is emphasized that this abstract is provided to comply with the rules requiring an abstract which will allow a searcher or other reader quickly to ascertain the subject matter of the technical disclosure. It is submitted with the understanding that it will not be used to interpret or limit the scope or meaning of the appended issued claims.

### BACKGROUND OF THE INVENTION

1. Field of the Invention

The present invention relates generally to ophthalmology.

2. Description of Related Art

Ophthalmic solutions intended to be used as treatments for eye conditions, such as dry eyes or inflammation, or in connection with contact lenses typically comprise a content of antimicrobial agents as preservatives. The use of such preservatives is fraught with problems, as the preservatives may react with therapeutically active ingredients also contained within the ophthalmic solution, or else may cause side-effects, or even allergies. Short-term applications of such ophthalmic solutions to the eye may not cause visible effects or significant damage to the eye, but where the patient is obliged to apply the ophthalmic solutions directly to the eye or to a contact lens to be placed on the cornea over longer periods of time, the eye may be visibly impaired and otherwise suffer significant damage.

Lysozyme is one of the major pertinacious components in human tears. Lysozyme is an enzyme that acts as an antimicrobial agent by degrading glycosidic linkages between N-acetylmuramic acid and N-acetylglucosamine units of the microbial cell wall. Thus, the presence of lysozyme in human tears is a natural defense mechanism against ocular infections.

However, for example, in the situation where the patient is suffering dry eye or inflammation, tears are not being produced at all or in their normal quantity and/or quality. In such situations, the normal protective effect of lysozyme is lost altogether or significantly reduced. It is perhaps for this reason that a number of recent disclosures of ophthalmic solutions include hen egg white lysozyme as a component. (Hen eggs are the leading source of manufactured lysozyme.) Thus, for example, WO 91 17469, the entire contents of which are hereby incorporated by reference, describes a lacrophyl solution for contact lenses comprising lysozyme, ascorbic acid and citric acid as actives. Similarly, U.S. Pat. No. 6,949,241, the entire contents of which are also incorporated herein by reference, describes a lacrophyl solution for eye drops comprising lysozyme and, optionally, therapeutic ingredients.

Because hen egg white lysozyme is not native human lysozyme, and because the administration directly into the eye is considered systemic, its use in such ophthalmic solutions may cause serious allergic reactions in at least those humans who are sensitive to egg proteins and who may use these ophthalmic solutions. In fact, it is estimated that infants and small children are the most sensitive to eggs (31%). While 8% of adults with food allergies are allergic to eggs of all egg allergic individuals, 35% have shown to have anti-lysozyme IgE. (“Prevalence of lysozyme sensitization in an egg-allergic population,” Fremont S. et al, Allergy. 1997 February; 52(2): 224-8.) Accordingly, there is a need for improved ophthalmic solutions that do not have this allergic potential.

### BRIEF SUMMARY OF THE INVENTION

These and other objects were met with the present invention, which relates in a first embodiment to an ophthalmic solution comprising:

a) a content of a human recombinant lysozyme;

b) one or more natural lacrophyl substances;

c) water; and

d) optionally one or more therapeutic substances.

The present invention relates in a second embodiment to a method of treating a patient suffering from an eye condition related to insufficient or inadequate tearing, such as dry eye, or to inflammation of the eye, wherein the method involves topically administering the inventive ophthalmic solution to the patient.

The present invention relates in a third embodiment to a method of conditioning and/or cleansing a contact lens by contacting the contact lens with the inventive ophthalmic solution for a period of time sufficient to condition and/or cleanse the contact lens.

### DETAILED DESCRIPTION OF THE INVENTION

The inventive ophthalmic solution comprises a content of human recombinant lysozyme. This material is known, and the details of its preparation are not repeated here. Reference is made, merely for example, to U.S. Pat. No. 6,991,824, the entire contents of which are hereby incorporated herein by reference. The content of human recombinant lysozyme in the inventive ophthalmic solution is an amount determined to provide effective antimicrobial action. In a preferred embodiment, the inventive ophthalmic solution comprises about 0.01 to about 5% by weight of human recombinant lysozyme. In an especially preferred embodiment, the inventive ophthalmic solution comprises about 0.1 to about 1% by weight of human recombinant lysozyme.

The presence of the human recombinant lysozyme in the inventive ophthalmic solution ensures that not only the inventive ophthalmic solutions themselves, but also the eyes to which they are applied, will be adequately protected from the action of microbes. Due the presence of the human recombinant lysozyme in the inventive ophthalmic solution, there is no need for the usual chemical preservatives, and, in a preferred embodiment, preservatives that are not substances naturally occurring in the human body are expressly excluded.

The inventive ophthalmic solution also comprises a content of one or more natural lacrophyl substances, for example, alpha-hydroxy acids, buffers, and chelating agents. In a preferred embodiment, the inventive ophthalmic solution comprises one or more natural lacrophyl substances selected from the group consisting of ascorbic acid, citric acid, boric acid and ethylenediaminetetraacetic acid and physiologically acceptable salts of said acids.

In one embodiment, the inventive ophthalmic solution comprises at least a content of ascorbic acid or a physiologically acceptable salt thereof. Preferably, the inventive ophthalmic solution comprises about 0.01 to about 15% by weight of ascorbic acid or a physiologically acceptable salt thereof. In an especially preferred embodiment, the inventive ophthalmic solution comprises about 0.02 to about 10% by weight of ascorbic acid or a physiologically acceptable salt thereof.

In another embodiment, the inventive ophthalmic solution comprises at least a content of citric acid or a physiologically acceptable salt thereof. Preferably, the inventive ophthalmic solution comprises about 0.01 to about 5% by weight of citric acid or a physiologically acceptable salt thereof. In an especially preferred embodiment, the inventive ophthalmic solution comprises about 0.01 to about 1% by weight of citric acid or a physiologically acceptable salt thereof.

In another embodiment, the inventive ophthalmic solution comprises at least a content of boric acid or a physiologically acceptable salt thereof. Preferably, the inventive ophthalmic solution comprises about 0.01 to about 5% by weight of boric acid or a physiologically acceptable salt thereof. In an especially preferred embodiment, the inventive ophthalmic solution comprises about 0.01 to about 1% by weight of boric acid or a physiologically acceptable salt thereof.

In another embodiment, the inventive ophthalmic solution comprises at least a content of ethylenediaminetetraacetic acid (EDTA) or a physiologically acceptable salt thereof. Preferably, the inventive ophthalmic solution comprises about 0.01 to about 1% by weight of ethylenediaminetetraacetic acid or a physiologically acceptable salt thereof. In an especially preferred embodiment, the inventive ophthalmic solution comprises about 0.01 to about 0.1% by weight of ethylenediaminetetraacetic acid or a physiologically acceptable salt thereof.

In a particularly preferred embodiment, the inventive ophthalmic solution comprises a content of all of ascorbic acid, citric acid, boric acid or EDTA or the physiologically acceptable salts thereof in the “preferred” and “especially preferred” ranges given above.

The bulk of the inventive ophthalmic solution is composed of water, preferably at least 75% by weight, especially at least 85% by weight. In a particularly preferred embodiment, the water is distilled water.

In addition to the foregoing ingredients, the inventive ophthalmic solution can have other ingredients well known in the art normally to be included in ophthalmic solutions generally.

Thus, for example, the inventive ophthalmic solution optionally comprises one or more compounds therapeutically active against eye disorders. Such compounds are present in the inventive ophthalmic solution in an amount which is empirically determined to be effective against the particular eye disorders against which the compounds are active. In a preferred embodiment, these therapeutically active compounds are selected from the group consisting of antibiotics, steroids and compounds for treating glaucoma.

In one embodiment, the inventive ophthalmic solution comprises one or more antibiotics selected from the group consisting of rifampicin, tetracycline and quinolone antibiotics. In an especially preferred embodiment, the inventive ophthalmic solution comprises a quinolone antibiotic selected from the group consisting of ciprofloxacin and physiologically acceptable salts thereof.

In another embodiment, the inventive ophthalmic solution comprises the steroid fluorometholone.

In another embodiment, the inventive ophthalmic solution comprises more or more compounds for treating glaucoma selected from the group consisting of pilocarpine, levobunolone, and timolol maleate and physiologically acceptable salts thereof.

In another embodiment, the inventive ophthalmic solution comprises other ingredients, for example, an eye diagnostic agent. In an especially preferred embodiment, the inventive ophthalmic solution comprises the eye diagnostic agent fluorescein-sodium.

The inventive ophthalmic solution may additionally comprise ingredients to help solubilize the recombinant human lysozyme and any therapeutic active compounds that may be present. In one embodiment, the inventive ophthalmic solution comprises tris(hydroxymethyl)aminomethane, preferably from about 0.01 to about 10% by weight of tris(hydroxymethyl)aminomethane. In an especially preferred embodiment, the inventive ophthalmic solution comprises from 0.1 to 5% by weight of tris(hydroxymethyl)aminomethane.

Where the inventive ophthalmic solution is intended to be used in connection with the wearing and/or cleansing of contact lenses, it is advantageous to include in the inventive ophthalmic solution a content of chitosan or a derivative thereof, especially an anionic chitosan. Anionic derivatives of chitosan are effective in removing protein deposits from contact lenses by means of ionic interactions with the lysozyme contained in those deposits. It is also known that chitosan derivatives herein enhance the lubricity of contact lenses and protect corneal epithelial cells from desiccation. All of these functions promote the ocular comfort of persons wearing contact lenses. The useful chitosans and details on their use in ophthalmic solutions of the type described herein are fully set forth in US 20040121924, the entire contents of which are hereby incorporated herein by reference. Generally, the chitosan derivatives used in the present invention include one or more anionic functional groups, such as sulfuryl chitosan, phosphoryl chitosan, carboxymethyl chitosan, dicarboxymethyl chitosan, and succinyl chitosan. The preferred chitosan derivative is carboxymethyl chitosan. The polymers have molecular weights ranging from 500 to 10,000,000 Daltons. The selection of an ideal molecular weight of a particular chitosan derivative and the desired viscosity of the composition can be readily determined by persons skilled in the art. The inventive ophthalmic solution of the present invention will, if they are used to clean contact lenses, will typically comprise one or more chitosan derivatives in an amount of from about 0.01 to 10% by weight, preferably about 0.1 to about 1% by weight.

The inventive ophthalmic solution can be used to treat dry eye conditions and eye inflammation, for example, ulcerative herpetic kerititis, in humans and animals. For this purpose, one or two drops (or more if directed by a physician) are dropped two to three times a day, or as needed, into the eye. Depending on the condition, this regiment will need to be followed for a period of days, weeks, months and perhaps even years.

The inventive ophthalmic solution can also be used to condition and/or cleanse contact lenses. For this purpose, the contact lens to be conditioned and/or cleansed is contacted with the inventive ophthalmic solution, usually by placing the contact lens in a container containing the inventive ophthalmic solution, and the contact lens is kept in the inventive ophthalmic solution for a period of minutes, hours or days, as needed, until the contact lens achieves the desired level of conditioning and/or cleansing.

The invention will now be described in greater detail with reference to the following non-limiting examples.

### EXAMPLES

### Example 1

A rifampicin eye-drop can be prepared as follows:

| Ingredients | Amount | |
|---|---|---|
| Rifampicin | 0.02 | g |
| Human recombinant lysozyme | 0.01 | g |
| citric acid | 0.16 | g |
| boric acid | 0.05 | g |
| EDTA-Na₂ | 0.01 | g |
| tris(hydroxymethyl)aminomethane | 0.01 | g |
| distilled water | ad 10 | ml |

### Example 2

A doxycycline hydrochloride eye-drop can be prepared as follows:

| Ingredients | Amount | |
|---|---|---|
| Doxycycline HCl | 0.05 | g |
| Human recombinant lysozyme | 0.01 | g |
| citric acid | 0.16 | g |
| boric acid | 0.05 | g |
| EDTA-Na₂ | 0.01 | g |
| tris(hydroxymethyl)aminomethane | 0.01 | g |
| distilled water | ad 10 | ml |

### Example 3

A pilocarpine chloride eye-drop can be prepared as follows:

| Ingredients | Amount | |
|---|---|---|
| Pilocarpine HCl | 0.1 | g |
| Human recombinant lysozyme | 0.01 | g |
| citric acid | 0.16 | g |
| boric acid | 0.05 | g |
| EDTA-Na₂ | 0.01 | g |
| tris(hydroxymethyl)aminomethane | 0.01 | g |
| distilled water | ad 10 | ml |

### Example 4

A fluorescein sodium eye-drop can be prepared as follows:

| Ingredients | Amount | |
|---|---|---|
| Fluorescein-sodium | 0.1 | g |
| Human recombinant lysozyme | 0.01 | g |
| citric acid | 0.16 | g |
| boric acid | 0.05 | g |
| EDTA-Na₂ | 0.01 | g |
| tris(hydroxymethyl)aminomethane | 0.01 | g |
| distilled water | ad 10 | ml |

### Example 5

A contact lens cleaning solution can be prepared as follows:

| Ingredients | Amount | |
|---|---|---|
| Human recombinant lysozyme | 0.01 | g |
| ascorbic acid | 0.02 | g |
| citric acid | 0.16 | g |
| boric acid | 0.05 | g |
| EDTA-Na₂ | 0.01 | g |
| tris(hydroxymethyl)aminomethane | 0.01 | g |
| carboxymethylchitosan | 0.5 | g |
| distilled water | ad 10 | ml |

It should be understood that the preceding detailed description of the invention is merely a detailed description of one preferred embodiment or of a small number of preferred embodiments of the present invention and that numerous changes to the disclosed embodiment(s) can be made in accordance with the disclosure herein without departing from the spirit or scope of the invention. The preceding detailed description of the invention, therefore, is not meant to limit the scope of the invention in any respect. Rather, the scope of the invention is to be determined only by the appended issued claims and their equivalents.

What is claimed is:

1. An ophthalmic solution comprising:
a) a content of a human recombinant lysozyme;
b) one or more natural lacrophyl substances;
c) water; and
d) optionally one or more therapeutic substances.

2. The ophthalmic solution according to claim 1, which comprises about 0.01 to about 5% by weight of human recombinant lysozyme.

3. The ophthalmic solution according to claim 2, which comprises about 0.1 to about 1% by weight of human recombinant lysozyme.

4. The ophthalmic solution according to claim 1, which comprises one or more natural lacrophyl substances selected from the group consisting of ascorbic acid, citric acid, boric acid and ethylenediaminetetraacetic acid and physiologically acceptable salts of said acids.

5. The ophthalmic solution according to claim 4, which comprises about 0.01 to about 15% by weight of ascorbic acid or a physiologically acceptable salt thereof.

6. The ophthalmic solution according to claim 5, which comprises about 0.02 to about 10% by weight of ascorbic acid or a physiologically acceptable salt thereof.

7. The ophthalmic solution according to claim 4, which comprises about 0.01 to about 5% by weight of citric acid or a physiologically acceptable salt thereof.

8. The ophthalmic solution according to claim 7, which comprises about 0.01 to about 1% by weight of citric acid or a physiologically acceptable salt thereof.

9. The ophthalmic solution according to claim 4, which comprises about 0.01 to about 5% by weight of boric acid or a physiologically acceptable salt thereof.

10. The ophthalmic solution according to claim 9, which comprises about 0.01 to about 1% by weight of boric acid or a physiologically acceptable salt thereof.

11. The ophthalmic solution according to claim 4, which comprises about 0.01 to about 1% by weight of ethylenediaminetetraacetic acid or a physiologically acceptable salt thereof.

12. The ophthalmic solution according to claim 11, which comprises about 0.01 to about 0.1% by weight of ethylenediaminetetraacetic acid or a physiologically acceptable salt thereof.

13. The ophthalmic solution according to claim 1, which comprises distilled water.

14. The ophthalmic solution according to claim 1, which comprises one or more therapeutically active compounds selected from the group consisting of antibiotics, steroids and compounds for treating glaucoma.

15. The ophthalmic solution according to claim 14, which comprises one or more antibiotics selected from the group consisting of rifampicin, tetracycline and quinolone antibiotics.

16. The ophthalmic solution according to claim 15, which comprises a quinolone antibiotic selected from the group consisting of ciprofloxacin and physiologically acceptable salts thereof.

17. The ophthalmic solution according to claim 14, which comprises the steroid fluorometholone.

18. The ophthalmic solution according to claim 14, which comprises more or more compounds for treating glaucoma selected from the group consisting of pilocarpine, levobunolone, and timolol maleate and physiologically acceptable salts thereof.

19. The ophthalmic solution according to claim 1, which further comprises an eye diagnostic agent.

20. The ophthalmic solution according to claim 19, wherein the eye diagnostic agent is fluorescein-sodium.

21. The ophthalmic solution according to claim 1, which further comprises from about 0.01 to about 10% by weight of tris(hydroxymethyl)aminomethane.

22. The ophthalmic solution according to claim 21, which further comprises from 0.1 to 5% by weight of tris(hydroxymethyl)aminomethane.

23. The ophthalmic solution according to claim 1, which further comprises a content of a chitosan.

24. A method of treating a patient suffering from a dry eye condition, said method comprising topically administering to said patient an effective amount therefor of an ophthalmic solution according to claim 1.

25. A method of treating a patient suffering from eye inflammation, said method comprising topically administering to said patient an effective amount therefor of an ophthalmic solution according to claim 1.

26. A method of conditioning and/or cleansing a contact lens, said method comprising contacting said contact lens with an ophthalmic solution according to claim 1 for a period of time sufficient to condition and/or cleanse said contact lens.
